(19) United States
(12) Patent Application Publication
(10) Pub. No.: US 2007/0202493 A1
(43) Pub. Date: Aug. 30, 2007

(54) Device and method for maintaining a plurality of beads in suspension

(76) Inventors: Aaron Mason, Wilmington, MA (US); Robert Burger, Newton, MA (US)

Correspondence Address: Patrick W. Rasche;Armstrong Teasdale LLP, Suite 2600, One Metropolitan Square, St. Louis, MO, 63102, US

(21) Appl. No.: 11/362,968

(22) Filed: Feb. 27, 2006

(51) Int. Cl. C12Q 1/70; G01N 33/53; G01N 33/569; G01N 33/554

(52) U.S. Cl. 435/5; 435/7.1; 435/7.31; 435/7.32; 422/73

(57) ABSTRACT

A device and a method for maintaining a plurality of beads in suspension in a fluid are provided. The device includes a container having a chamber in fluid communication with an inlet and an outlet. The chamber is configured to hold the plurality of beads and the fluid therein. Each bead of the plurality of beads has at least one antibody disposed thereon. The inlet is configured to route at least one antigen into the chamber. The outlet is configured to route the plurality of beads out of the chamber. The device further includes a mounting member configured to secure the container thereon. The device further includes a voice coil actuator operably coupled to the mounting member and to a base member. The voice coil actuator is configured to provide reciprocating linear movement to the mounting member and to the container secured on the mounting member to induce the plurality of beads to be suspended in the fluid such that the at least one antigen reacts with the at least one antibody of the plurality of beads.

### BACKGROUND

A device and a method for maintaining a plurality of beads in suspension in a fluid are provided.

An antigen detection system has utilized a container having a plurality of beads coated with antibodies to detect specific antigens. The plurality of beads are disposed in a fluid within the container. When an antigen is present in the fluid, the antigen may react with the antibodies if the antigen contacts the antibodies. A problem associated with the antigen detection system, however, is that the plurality of beads settle at the bottom of the container over time. Thus, antigens in the fluid may pass through the container without contacting the antibodies on the plurality of beads disposed at the bottom of the container, resulting in the antigens not being detected.

Accordingly, it is desirable to have an improved system and method that maintains a plurality of beads in suspension in a fluid.

### SUMMARY OF THE INVENTION

A device for maintaining a plurality of beads in suspension in a fluid in accordance with an exemplary embodiment is provided. The device includes a container having a chamber in fluid communication with an inlet and an outlet. The chamber is configured to hold the plurality of beads and the fluid therein. Each bead of the plurality of beads has at least one antibody disposed thereon. The inlet is configured to route at least one antigen into the chamber. The outlet is configured to route the plurality of beads out of the chamber. The device further includes a mounting member configured to secure the container thereon. The device further includes a voice coil actuator operably coupled to the mounting member and to a base member. The voice coil actuator is configured to provide reciprocating linear movement to the mounting member and to the container secured on the mounting member to induce the plurality of beads to be suspended in the fluid such that the at least one antigen reacts with the at least one antibody of the plurality of beads.

A method for maintaining a plurality of beads in suspension in a fluid in accordance with another exemplary embodiment is provided. The method includes reciprocatively linearly moving a container having a plurality of beads disposed in the fluid therein, such that the plurality of beads are suspended in the fluid and the at least one antigen in the fluid reacts with the at least one antibody disposed on the plurality of beads.

The above described and other features are exemplified by the following figures and detailed description.

### BRIEF DESCRIPTION OF THE DRAWINGS

FIG. 1 is a perspective view of a device for agitating a fluid having a container, a mounting member, and a voice coil actuator in accordance with an exemplary embodiment;

FIG. 2 is a top view of the container of the device of FIG. 1;

FIG. 3 is a side view of the mounting member, the voice coil actuator, and the base member of the device of FIG. 1;

FIG. 4 is a prospective view of the mounting member, and the base member of the device of FIG. 1; and

FIG. 5 is a cross-sectional view of the voice coil actuator of the device of FIG. 1.

### DETAILED DESCRIPTION OF EXEMPLARY EMBODIMENTS

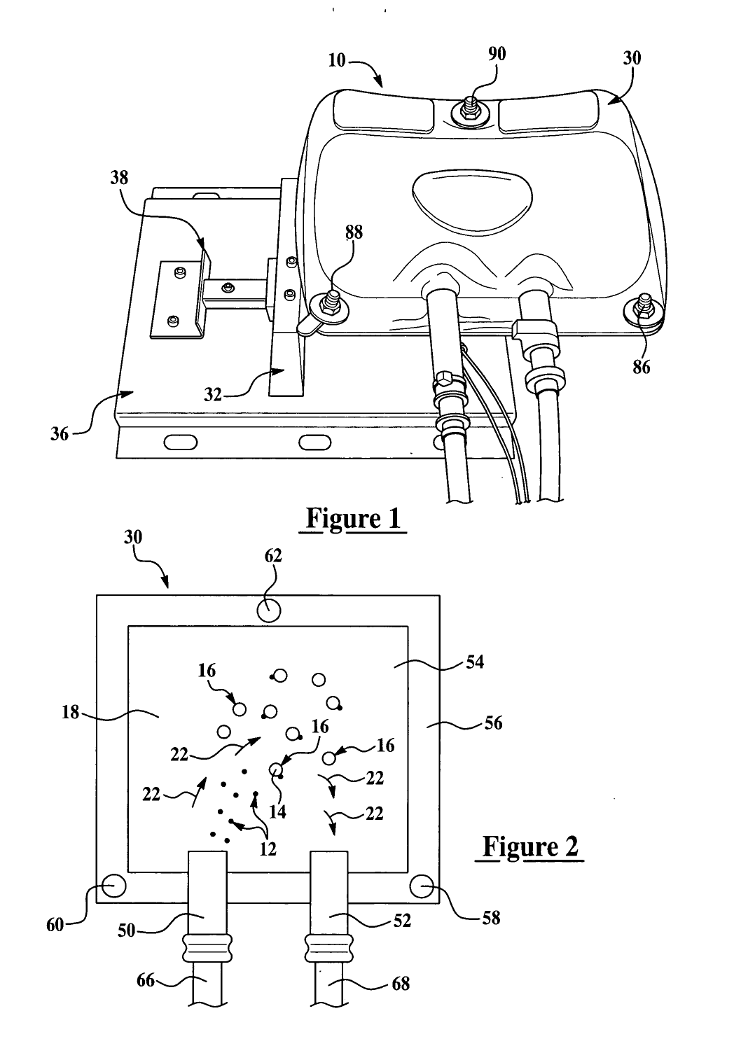

Referring to FIGS. 1-5, a device 10 for agitating a fluid 18 disposed in a container 30 is shown. By agitating the fluid in the container 30, the device 10 can maintain a plurality of beads 16 in suspension in fluid 18. When suspended in fluid 18, antigen 12 disposed in fluid 18 can more effectively react with antibodies 14 disposed on plurality of beads 16. The device 10 includes the container 30, a mounting member 32, a voice coil actuator 34, a base member 36, and a sliding member 38.

Referring to FIG. 2, container 30 is provided to hold plurality of beads 16 and fluid 18 such that the antigen 12 can react with one or more antibodies 14 disposed on plurality of beads 16. In an exemplary embodiment, container 30 is a plastic bag configured to hold plurality of beads 16 and fluid 18 therein. In an alternative exemplary embodiment, container 30 is a rigid plastic container or a glass container. Container 30 can be transported from a contaminant free clean room or other controlled environment. Thereafter, container 30 can be secured mounting member 32 of device 10. Container 30 and can be replaced by another container at an end of an operational life of the plurality of beads 16. Container 30 includes an outer peripheral wall 56 defining a chamber 54 and has an inlet 50 and an outlet 52 in fluid communication with the chamber 54.

Inlet 50 is configured to route at least one antigen 12 into chamber 54. Inlet 50 is configured to receive an input hose 66 therein. Input hose 66 can route antigen 12 into the inlet 50. Antigen 12 can include any substance capable of reacting with antibodies, including pollen, proteins, viruses, bacteria, mold or other fungi, and metabolites. It should be noted that metabolites can comprise sugar or drugs disposed in blood or urine.

Outlet 52 is configured to allow plurality of beads 16 and fluid 18 to exit container 30. Outlet 52 is further configured to receive an output hose 68 therein. Output hose 68 provides a fluid path to route plurality of beads 16 and fluid 18 exiting container 30 to an antigen analysis device (not shown). The antigen analysis device can classify the antigen 12 by determining which of the antibodies 14 reacted with the antigen 12.

Outer peripheral wall 56 defines the chamber 54 holding the plurality of beads 16 and fluid 18. Outer peripheral wall 56 has an airtight seal so that fluid 18 and plurality of beads 16 are maintained in chamber 54 during the operation of the device 10. Outer peripheral wall 56 has holes 58, 60, and 62 disposed therethrough, but holes 58, 60, and 62 do not breach the seal of the chamber 54, in that the holes are not in fluid communication with chamber 54.

In an exemplary embodiment, plurality of beads 16 are constructed from a polystyrene material. In alternative embodiments, plurality of beads 16 are constructed from other plastics such as polymethacrylate, latex, and the like. Different sets of the plurality of beads 16 can have different antibodies 14 disposed thereon, wherein each antibody is configured to detect a different antigen. Referring to FIG. 2, plurality of beads 16 and antigen 12 are represented as being larger than their actual size relative to container 30 for illustration purposes. Although the device 10 can be adapted to utilize beads of various, shapes and sizes, in an exemplary embodiment, each bead is substantially spherical. Further, each bead has an average diameter of about 1 micrometer to about 50 micrometers and more specifically about 4 micrometers to about 7 micrometers as measured by taking the largest diameter across a bead. However, in an alternative exemplary embodiment, device 10 can be adapted to utilize beads of various other sizes. Plurality of beads 16 are disposed within container 30 at a location remote from device 10.

Antibodies 14 are provided to react with antigen 12 disposed in fluid 18. Antibodies 14 can be chosen to be specifically sensitive to a specific antigen such as specific types of pollen, proteins, viruses, bacteria, mold or other fungi, and metabolites or the like. The antibodies 14 can be applied to the plurality of beads 16 by coating the plurality of beads 16 with antibodies and/or injecting the antibodies into the beads 16.

Referring to FIGS. 1, 3, and 4, mounting member 32 is provided to secure container 30 thereon. Mounting member 32 includes a mounting plate 70, a bracket member 72 secured to the mounting plate 70 and an arch portion 76 disposed on the mounting plate 70. Mounting plate 70 has holes 80, 82, and 84 extending, therethrough. Holes 80, 82, and 84 are configured to align with the holes 58, 60, and 62, respectively, of the container 30 and are configured to receive securing members 86, 88, and 90 therethrough to secure the container 30 to mounting member 32. In an exemplary embodiment, securing members 86, 88, and 90 include nuts secured onto bolts The container 30 and the mounting plate 70 are disposed between a nut and a head of a bolt to secure the container 30 to the mounting member 32. However, in an alternative exemplary embodiment, container 30 can be secured to the mounting member 32 by other types of securing devices such as hook type fasteners, snapping fasteners, other mechanical fasteners, magnetic devices, or adhesive devices.

Bracket member 72 is provided to secure mounting plate 70 to voice coil actuator 34. Bracket member 72 is a metal casing having an upper member 100, a lower member 102, a front wall 104, a side wall 98, and another side wall (not shown). The upper member 100 and the lower member 102 are perpendicular to front wall 104 and are formed at opposite ends of front wall 104. Upper member 100 is secured to mounting plate 70. The side walls are disposed at side edges of upper member 100 and the front wall 104, and provide structural support to the bracket member 72.

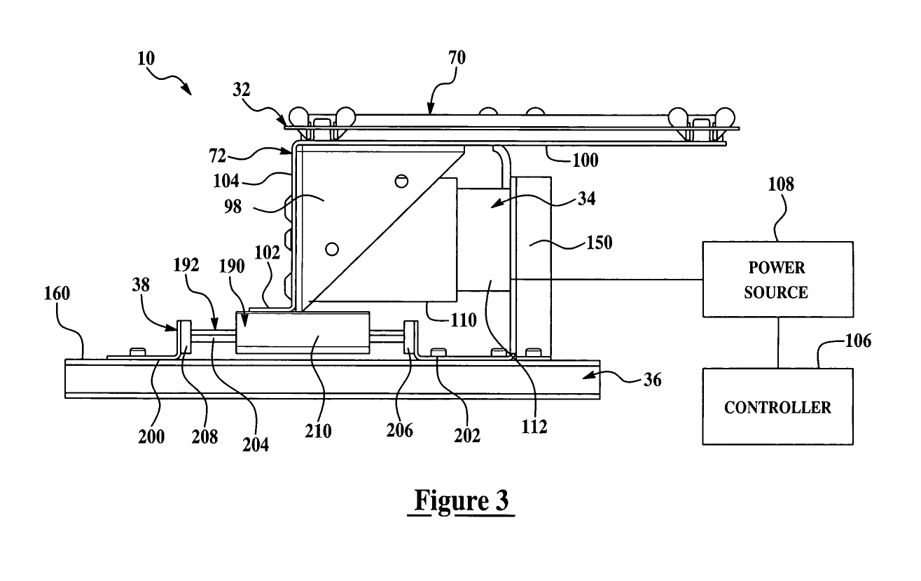

Referring to FIGS. 3 and 5, voice coil actuator 34 is provided to supply reciprocating linear movement to the container 30 to maintain the beads 16 in suspension in the fluid 18. Voice coil actuator 34 includes a housing member 110, a housing member 112, a core 114, a magnet member 116, and an electromagnetic coil 118.

In an exemplary embodiment, housing member 110 and housing member 112 are concentric, hollow cylindrical members, and housing member 110 is configured to receive a portion of the housing member 112. However, in alternative exemplary embodiments housing member 112 is configured to receive a portion of housing member 110 or housing members can be configured to move in positions relative to one another, without one housing member necessarily being received in the other housing member. Housing member 110 includes a top portion 120 that is secured to the front wall 104 of the bracket member 72. Housing member 112 is secured to an arm member 150 of base member 36. Housing member 110 and housing member 112 are made of ferromagnetic material such as steel, so as not to interfere with magnetic fields of voice coil actuator 34.

Core 114 is a cylindrical member attached on the inside of the top portion 120 of housing member 110 extending along an axis 130. Core 114 is made from steel, so as not to interfere with the magnetic fields produced by voice coil actuator 34. In other exemplary embodiments, core 114 can be made of other ferromagnetic materials.

Magnet member 116 is provided to create a magnetic field inside voice coil actuator 34. In an exemplary embodiment, magnet member 116 includes multiple magnets disposed inside housing member 110 of the voice coil actuator 34 extending approximately parallel to the core 114. Magnet member 116 is configured to create a magnetic field perpendicular to the axis 130. In other embodiments, the magnet member 116 could be disposed in other locations proximate the electromagnetic coil, so long as the magnet member 116 provides an electromagnetic field substantially perpendicular to the axis 130.

Electromagnetic coil 118 is provided to move housing member 110 relative to the housing member 112 at a selected frequency when the electromagnetic coil 118 receives electrical current. Electromagnetic coil 118 includes a wire 132 made of electrically conductive material that is wrapped around a spool 134. Spool 134 movably couples the electromagnetic coil to the core 114 and is secured to housing member 112. Spool 134 is made from an electrically insulative material so as not to conduct electricity between electromagnetic coil 118 and core 114. The spool has a hole 140 disposed therethrough, allowing the electromagnetic coil 118 to be slideably received on the core 114, and allowing electromagnetic coil 118 to move along the core, linearly in a first direction and in a second direction.

Electrical current is supplied to the electromagnetic coil 118 from an external power source 108 through lead wires 136 and 138 attached at each end of wire 132. As the electrical current travels through the electromagnetic coil 118, the electromagnetic field exerts a force on the electromagnetic coil 118 perpendicular to the electromagnetic field. The power source 108 supplies alternating electrical current to the electromagnetic coil 118 causing electromagnetic coil 118 to reciprocatively linearly move in the first and second directions. When the electrical current is supplied to the electromagnetic coil 118 in a first current direction, a generated electromagnetic field induces the electromagnetic coil to move in the first direction relative to the core. When the electrical current is supplied to the electromagnetic coil 118 in a second current direction, a generated electromagnetic field induces the electromagnetic coil 118 to move in the second direction, opposite the first direction.

When the electromagnetic coil 118 moves in the first direction, the electromagnetic coil along with the housing member 112 moves toward the top portion 120 of the housing member 110. When the electromagnetic coil 118 moves in the second direction, the electromagnetic coil 118 along with the second housing member 112 move away from the top portion 120. Therefore, as electromagnetic coil 118 travels in the first direction and in the second direction along core 114, the electromagnetic forces move the housing member 110 relative to housing member 112. During the operation of the voice coil actuator 34, the relative movement of the housing member 110 and the housing member 112 moves mounting member 32.

A controller 106 is provided to control the electrical current supplied to electromagnetic coil 118, thereby controlling movement of electromagnetic coil 118. The controller 106 operably communicates with a power supply 108 and can generate a first signal to induce the power supply 108 to supply electromagnetic coil 118 with alternating electrical current at a first predetermined frequency for a first predetermined amount of time. In response to the alternating electrical current at the first predetermined frequency, electromagnetic coil 118 moves reciprocatively linearly relative to the core 114. The movement of the electromagnetic coil 118 provides movement to the mounting member 32 and the container 30 secured on the mounting member 32 at a first frequency corresponding to the frequency of the electrical current. The movement of the mounting member 32 and the container 30 induce the plurality of beads 16 to be suspended in the fluid such that at least one antigen 12 reacts with at least one antibody 14 of the plurality of beads 16. Further, the controller 106 can generate other signals corresponding to other frequencies. For example, the controller 106 can generate a second signal to induce the power supply 108 to supply the electromagnetic coil 118 with alternating current at a second predetermined frequency to move the mounting member 32 and the container 30 reciprocatively linearly at a second frequency for a second predetermined amount of time. Further, the controller 106 can generate various other signals to induce power supply 108 to supply electromagnetic coil 118 with current at various frequencies to reciprocatively move mounting member 32 at various frequencies over various time periods.

It should be noted that the device 10 can utilize a voice coil actuator 34 capable of moving the mounting member at vibrational frequencies that are selected according to specific functional requirements of device 10. The requirement can vary depending on the size and type of the beads, the size and type of the containers, and the type of antigen. In an exemplary embodiment, voice coil actuator 34 can move the mounting member at a frequency of about 10 Hertz to about 10,000 Hertz.

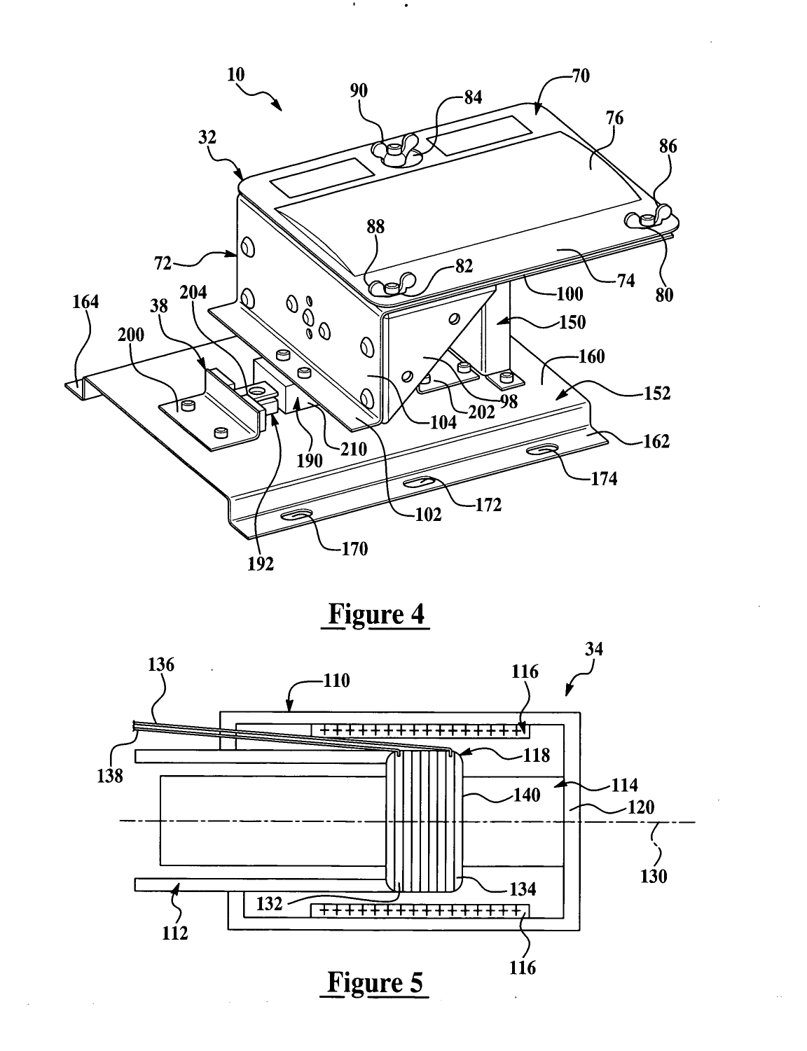

Referring to FIGS. 3 and 4, base member 36 is provided to support the mounting member 32 and the voice coil actuator 34. Base member 36 includes an arm member 150 secured to a base plate 152. Base plate 152 includes a platform 160 disposed between mounting rails 162 and 164. Arm member 150 is secured to the platform 160. Mounting rail 162 includes mounting holes 170, 172, and 174, and the mounting rail 134 includes mounting holes (not shown). Securing members (not shown) can be disposed through each of the mounting holes 170, 172, 174 (as well as other mounting holes) to secure the base member 36 to a support apparatus (not shown).

Arm member 150 extends perpendicularly from a portion of the platform 160. Arm member 150 is also secured to the second housing member 112 of the voice coil actuator 34, which extends perpendicularly from a side of arm member 150.

Sliding member 38 is provided to steady voice coil actuator 34 and mounting member 32 during movement. Sliding member 38 includes a sliding carriage 190 that is slideably coupled to a slide rail 192.

Slide rail 192 includes support members 200 and 202 secured to platform 160 of the base member 36, and a rail member 204 secured to platform 160 and each of support members 200 and 202. Support members 200 and 202 provide stabilizing support to the rail member across the plain of the platform 160. Slide rail 192 can further include barriers 206 and 208. Barriers 206 and 208 are formed of resilient material and are provided to limit forces induced by the sliding carriage 190.

Sliding carriage 190 includes an outer housing 210 and bearings (not shown) disposed inside the outer housing. Outer housing 210 is secured to lower member 102 of bracket member 72 of mounting member 32. The bearings are received in two parallel tracks of the rail member 204, and the two parallel tracks are configured to guide the movement of the sliding carriage 190 along slide rail 192. During operation of sliding member 38, the voice coil actuator 34 reciprocatively linearly moves mounting member 32 thereby reciprocatively linearly moving sliding carriage 192 attached to mounting member 32. Sliding carriage 190 moves in the first and second directions along slide rail 192.

Device 10 can maintain the plurality of beads 16 in suspension by operating continuously or by operating for discrete time periods. The device 10 can maintain the plurality of beads 16 in suspension by moving the mounting member at a single vibrational frequency. Further, the device 10 can maintain the plurality of beads 16 in suspension by moving the mounting member 32 at varying the vibrational frequency so that the device 10 will operate at different vibrational frequencies over different time periods.

Referring to FIG. 2, the operation of device 10 will now be described. Antigen 12 is routed through input hose 66 and inlet 50 into chamber 54. The antigen 12 disperses through the chamber 54 as represented by antigen flow path arrows 22 until antigen 12 contacts the plurality of beads 16 suspended in fluid 18. Since plurality of beads 16 is suspended in fluid 18, antigen 12 contacts the outer surfaces of beads 16. The antigen 12 then reacts with the antibodies 14. After a predetermined time period in which the antigen 12 is allowed to diffuse through the fluid 18 and contact the plurality of beads 16, the plurality of beads 16 and the fluid 18 is evacuated through outlet 52 and transported to the antigen analysis device (not shown) through output hose 68. Since different antigens can react with different antibodies, the antigen analysis device can classify the antigen 12 by determining which of the antibodies 14 reacted with the antigen 12. Each bead of the plurality of beads 16 can further comprise an internal dye that can indicate if the antigen 12 has reacted with the antibodies disposed on the bead. For example, the antigen analysis device can utilize a laser to excite the internal dyes that identify a specific bead coated with a specific antibody, and use the bead to classify the antigen 12.

The device 10 provides a technical effect of reciprocatively moving a container having a plurality of beads disposed in a fluid therein, such that the plurality of beads are suspended in the fluid and the at least one antigen in the fluid reacts with the at least one antibody disposed on the plurality of beads.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A device for maintaining a plurality of beads in suspension in a fluid comprising:
a container having a chamber in fluid communication with an inlet and an outlet, the chamber being configured to hold the plurality of beads and the fluid therein, each bead of the plurality of beads having at least one antibody disposed thereon, the inlet being configured to route at least one antigen into the chamber, the outlet being configured to route the plurality of beads out of the chamber;
a mounting member configured to secure the container thereon; and
a voice coil actuator operably coupled to the mounting member and to a base member, the voice coil actuator being configured to provide reciprocating linear movement to the mounting member and to the container secured on the mounting member to induce the plurality of beads to be suspended in the fluid such that the at least one antigen reacts with the at least one antibody of the plurality of beads.

2. The device of claim 1, wherein the voice coil actuator is configured to reciprocatively move the mounting member at a frequency in a range of 10 Hertz to 10,000 Hertz.

3. The device of claim 1, wherein the plurality of beads are a plurality of polystyrene beads.

4. The device of claim 1, wherein the plurality of beads are a plurality of polymethacrylate beads.

5. The device of claim 1, wherein the plurality of beads are a plurality of latex beads.

6. The device of claim 1, wherein the each bead of the plurality of beads has an average diameter in a range of 1 micrometer to 50 micrometers.

7. The device of claim 1, wherein the antigen comprises at least one of a pollen, a protein, a virus, a bacterium, a fungus, and a metabolite.

8. The device of claim 1, wherein the container is a plastic bag configured to hold the plurality of beads and the fluid therein.

9. The device of claim 1, wherein the mounting member is configured to secure the container thereon utilizing one or more securing members.

10. A method for maintaining a plurality of beads in suspension in a fluid, the method comprising:
reciprocatively linearly moving a container having a plurality of beads disposed in the fluid therein, such that the plurality of beads are suspended in the fluid and the at least one antigen in the fluid reacts with the at least one antibody disposed on the plurality of beads.

11. The method of claim 10, further comprising:
receiving a first signal at a voice coil actuator that induces the voice coil actuator to reciprocatively linearly move a mounting member coupled to the container at a first frequency.

12. The method of claim 11 wherein the first frequency is in a range of 10 Hertz to 10,000 Hertz.

13. The method of claim 11, further comprising:
receiving a second signal at the voice coil actuator that induces the voice coil actuator to reciprocatively linearly move the mounting member coupled to the container at a second frequency greater than the first frequency.

14. The method of claim 13, wherein the second frequency is in a range of 10 Hertz to 10,000 Hertz.

15. The method of claim 10, wherein the antigen comprises at least one of a pollen, a protein, a virus, a bacterium, a fungus, and a metabolite.
